(19) United States
(12) Patent Application Publication
(10) Pub. No.: US 2006/0057069 A1
(43) Pub. Date: Mar. 16, 2006

(54) Detection of protein expression in vivo using fluorescent puromycin conjugates

(75) Inventors: Shelley R. Starck-Green, Berkeley, CA (US); Harry M. Green, Berkeley, CA (US); Jose Alberola-ila, Altadena, CA (US); Richard W. Roberts, South Pasadena, CA (US); Erin Schuman, Pasadena, CA (US); William Bryan Smith, La Jolla, CA (US); Bruce A. Hay, Los Angeles, CA (US)

Correspondence Address: DLA PIPER RUDNICK GRAY CARY US, LLP, 4365 EXECUTIVE DRIVE, SUITE 1100, SAN DIEGO, CA, 92121-2133, US

(73) Assignee: CALIFORNIA INSTITUTE OF TECHNOLOGY, Pasadena, CA (US)

(21) Appl. No.: 11/147,813

(22) Filed: Jun. 7, 2005

Related U.S. Application Data
(60) Provisional application No. 60/577,903, filed on Jun. 7, 2004.

(51) Int. Cl. A61K 49/00; C07K 14/47

(52) U.S. Cl. 424/9.6; 530/352

(57) ABSTRACT

Disclosed is a class of reagents for examining protein expression in vivo that does not require transfection, radiolabeling, or the prior choice of a candidate gene. Further, a series of puromycin conjugates was constructed bearing various labeling moieties. These conjugates were readily incorporated into expressed protein products in cell lysates in vitro and efficiently cross cell membranes to function in protein synthesis in vivo as indicated by flow cytometry, selective enrichment studies, and western analysis. The present invention demonstrates that labeled-puromycin conjugates offer a general means to examine protein expression in vivo.

This application claims priority from U.S. Provisional Application Ser. No. 60/577,903 filed Jun. 7, 2004, the entire contents of which is incorporated herein by reference.

This invention was made in part with government support under Grant No. R01 GM 60416 awarded by the National Institutes of Health. The government has certain rights in this invention.

### BACKGROUND OF THE INVENTION

### FIELD OF THE INVENTION

The present invention relates generally to labeling proteins, and more specifically to incorporating puromycin conjugates bearing various moieties into expressed protein products.

### BACKGROUND INFORMATION

Complete sequencing of the human genome [1,2] shows that less than 50% of the putative gene transcripts correspond to known proteins. A complete understanding of the proteome awaits the identification of thousands of unassigned gene products and assignment of their role in signaling cascades [3], membrane trafficking [4], apoptosis [5], and other cellular processes. Currently, there are large-scale techniques to study cellular protein levels indirectly using DNA and mRNA arrays [6]. However, these techniques do not directly monitor the level of protein synthesis. Methods to directly monitor protein expression in vivo are extremely useful, particularly in the study of higher organisms with many different cell and tissue types.

Currently, protein expression is studied using pulse-labeling with a radioactive tracer or by transformation with fluorescent reporters based on the green fluorescent protein (GFP) and mutants (BFP, CFP, and YFP) [7]. Pulse-labeling experiments typically require the cell(s) to be destroyed and are not amenable to microscopy experiments with simultaneous protein synthesis detection. Genetically encoded GFP mutants and fusion proteins have seen broad biological applications including study of Ca²⁺ localization [8] protein tyrosine kinase activity [9], and mRNA trafficking and protein synthesis localization in cultured neurons [10,11]. However, the use of GFP-based constructs is limited to cells that can be efficiently transfected. Additionally, DNA transfection protocols often require several days to produce cells yielding robust GFP-based fluorescent signals and also inundate the protein synthesis machinery with a non-native transcript due to the use of strong upstream promoters. Finally, transfection-based strategies generally require choice of a particular candidate gene product.

In view of these shortcomings, puromycin-based reagents might provide a general means to examine protein expression. Puromycin is a structural analogue of aminoacylated-tRNA (aa-tRNA) and participates in peptide-bond formation with the nascent polypeptide chain (FIG. 1A) [12,13]. Previously, various puromycin derivatives of the form X-dC-puromycin have been examined and shown to be functional during in vitro translation experiments [14-17 and U.S. Pat. No. 6,228,994]. In principle, a fluorescent or biotinylated variant of puromycin should be functional in protein synthesis in vivo if it is able to enter cells in a non-destructive fashion (FIG. 1B). In this way, selective labeling of newly synthesized proteins would enable direct monitoring of protein expression and provide the potential for both spatial and temporal resolution.

The present invention satisfies this need, as well as others.

### SUMMARY OF THE INVENTION

The present invention demonstrates that a variety of puromycin conjugates can be used as detectors of protein synthesis in live cells. Further, the instant disclosure shows that puromycin conjugates can easily enter cells and covalently label newly synthesized proteins, enabling direct detection of protein expression in vivo.

In one embodiment, a labeled protein including a C-terminal chemically linked to a conjugate, where the conjugate comprises puromycin, puromycin derivative, a phosphonate-puromycin-aminonucleoside-R₃ compound, or a phosphonate-puromycin-aminonucleoside-R₃ compound derivative linked to at least one label moiety and where R₃ is an amino acid or amino acid analog is envisaged. In a related aspect, the phosphonate-puromycin-aminonucleoside-R₃ compound or a phosphonate-puromycin-aminonucleoside-R₃ compound derivative is of the general Formula I:

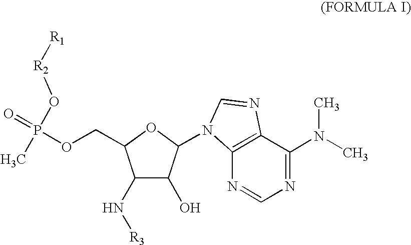

where R₁ is one or more label moieties; R₂ is a nucleotide; and R₃ is:

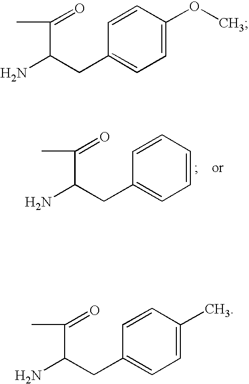

Moreover, in one aspect, R₃ is:

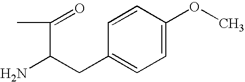

Further, R₂ can be a ribonucleotide or deoxyribonucleotide, for example, R₂ can be deoxycytidine-5′-monophosphate.

In a related aspect, R₁ includes a fluorescent substance, biotin, protein, peptide, nucleic acid, sugar, lipid, or dye. Further, the label moiety may include a dimethoxytrityl (DMT) moiety and/or two or more label moieties.

In another embodiment, a method of monitoring protein expression is envisaged, including contacting a sample having the minimum components necessary for protein translation with a conjugate having puromycin, puromycin derivative, a phosphonate-puromycin-aminonucleoside-R₃ compound, or a phosphonate-puromycin-aminonucleoside-R₃ compound derivative linked to at least one label moiety, where R₃ is an amino acid or an amino acid analog, under conditions that allow for protein translation and determining the presence of label incorporated into protein after a sufficient time, where incorporation of label into protein is correlated with protein expression.

Further, the sample is an in vitro translation extract or a cell, and where the sample is a cell, the cell may be transfected with a fluorescence based reporter vector. Moreover, incorporation may be determined by chromatograpy, blotting, spectrometry, microscopy, flow cytometry, imaging, immunochemistry, or combinations thereof. In a related aspect, the methods resolves temporal protein expression and/or spatial protein expression.

In another related aspect, the structure of the conjugate is X—N-puromycin, X—N-puromycin derivative, an X—N-phosphonate-puromycin-aminonucleoside-R₃ compound, or an X—N-phosphonate-puromycin-aminonucleoside-R₃ compound derivative where R₃ is an amino acid or an amino acid analog, which X is a label moiety and N is a ribonucleotide or deoxyribonucleotide, where the conjugate has an IC₅₀ range from between about less than 1 μM to about 30 μM. In a related apsect, such conjugates may include salts thereof.

In one embodiment, a method of identifying a protein modulated by an exogenous agent is envisaged, including contacting an exogenous agent with a sample having the minimum components necessary for protein translation, contacting the sample with a conjugate having a puromycin, puromycin derivative, a phosphonate-puromycin-aminonucleoside-R₃ compound, or a phosphonate-puromycin-aminonucleoside-R₃ compound, where R₃ is an amino acid or an amino acid analog, linked to at least one label under conditions that allow for protein translation, determining the presence of label incorporated into a protein after a sufficient time, and comparing protein incorporation patterns in the presence and absence of the exogenous agent, where changes in incorporation of label for a protein in the sample in the presence and absence of the exogenous agent correlate with modulation of such protein by the agent.

In a related aspect, the exogenous agent is a mineral, ion, gas, light, sound, small molecule, agonist, antagonist, amino acid, ligand, receptor, protein, peptide, antibody, nucleic acid, lipid, carbohydrate, cell, tissue, virus, organ, bodily fluid, buffer, media, conditioned media, temperature, pressure or a combination thereof

In another embodiment, a conjugate is envisaged, including a puromycin or puromycin-derivative linked to at least two label moieties and/or containing a phosphonate linking group on the puromycin or puromycin-derivative. In a related aspect, the puromycin derivative may be a phosphonate-puromycin-aminonucleoside-R₃ compound, or a phosphonate-puromycin-aminonucleoside-R₃ compound, where R₃ is an amino acid or an amino acid analog.

In one embodiment, a kit is envisaged, including a conjugate having a puromycin, puromycin-derivative, phosphonate-puromycin-aminonucleoside-R₃ compound or a phosphonate-puromycin-aminonucleoside-R₃ compound derivative linked to at least one label moiety, instructions containing method steps for practicing identifying a protein modulated by an exogenous agent, monitoring protein expression, labeling a protein at a C-terminal, or a combination thereof, and a container comprising reagents necessary for carrying out the methods. In a related aspect, the kit includes a fluorescence based vector.

In a related aspect, the kit includes a fluorescence based vector.

Exemplary methods and compositions according to this invention, are described in greater detail below.

### BRIEF DESCRIPTION OF THE DRAWINGS

FIG. 1. (A) Puromycin (P) participates in peptide bond formation with the nascent polypeptide chain. (B) Puromycin-dye conjugates, of the form X-dC-puromycin where X=fluorescein (F), are also active in translation and become covalently linked to protein.

FIG. 2. (A) Structure of puromycin conjugates and negative control conjugates. (B) Structure of phosphonate-based puromycin and negative control conjugates.

FIG. 3. In vitro IC₅₀ determination for various puromycin conjugates. (A) Percent of globin translation relative to the no conjugate control for compounds 1, 2, 3, 4, 6, 8, 10, 11, 12. (B) Tricine-SDS-PAGE analysis of globin translation reactions in the presence of Cy52P(1) (top) and Cy52A(2) (bottom): Lane 1, no template and no conjugate; lane 2, globin alone; lanes 3-10, conjugate concentrations from 0.5 μM to 120 μM.

FIG. 4. Protein labeling with the fluorescent puromycin conjugate FB2P (3). (A) Tricine-SDS-PAGE analysis of globin translation reactions incubated with increasing concentrations of FB2P (1): Lane 1, no template, no conjugate; lane 2, globin alone; lane 3, 7 μM; lane 4, 35 μM; lane 5, 70 μM; lane 6, 140 μM; and lane 7, 210 μM. (B) Neutravidin-purified globin-FB2P complexes from translation reactions in (A).

FIG. 5. Analysis of puromycin conjugate activity in 16610D9 thymocyte cells. Dose-response analysis of 16610D9 thymocyte cells treated with F2P or F2A at (A) 5 μM and (B) 25 μM. Incubation times are 1 (▪), 7 (▪), 24 (□), and 48 h (▪). Untreated cells incubated for 1 h are indicated with (▪). Cells were analyzed using a flow cytometer and gated on a live cell population according to forward and side scatter plots. (C) Flow cytometry analysis of untreated cells (▪); Fluorescein-puromycin, FP (▪); F2P, 4 (□); F2P-Me, 10 (□); FB2P, 1 (□); BF2P, 8 (▪); DMT-F2P-Me, 12 (▪). Cells were incubated for 24 h with puromycin conjugates at 50 μM. Analysis was performed using flow cytometry using a live cell gate as in A and B. (D) Epi-fluorescence microcopy of D9 cells treated with DMT-F2P-Me (25 μM) with 200× magnification.

FIG. 6. Fluorescence shift analysis for puromycin conjugates versus negative control molecules in 16610D9 thymocyte cells. (A) Untreated cells (▪); BF2A, 9 (▪); BF2P, 8 (□). (B) Untreated cells (▪); DMT-F2A-Me, 12 (□); DMT-F2P-Me, 12 (□). Analysis was performed using flow cytometry using a live cell gate as described for FIG. 5.

FIG. 7. Mechanism of action of puromycin in 16610D9 thymocyte cells infected with MIG (A) and MIGPAC (B) constructs. Cells infected with MIG are sensitive (C) and MIGPAC are resistant (D) to puromycin action.

FIG. 8. Mechanism of action of puromycin conjugates in 16610D9 thymocyte cells. Cells infected with (A) MIG or (B) MIGPAC were treated with biotinylated-puromycin conjugates B2A (7) and B2P (6).

FIG. 9. Western analysis of 16610D9 thymocyte cells treated with a puromycin conjugate and analyzed using an a-fluorescein antibody: Lane 1, untreated cells; lane 2, BF2P, 8 (25 μM); lane 3, BF2A, 9 (25 μM); and anisomycin (250 ng/mL). Ponceau S stain was used to confirm equal protein loading. BF2P-conjugated protein is seen at many molecular weights indicating that the conjugate could target all translating ribosomes.

FIG. 10. Dopamine D1/D5 receptor activation stimulates protein synthesis in hippocampal neurons. A, P2 cultured hippocampal neurons infected with a sindbis virus encoding a GFP reporter. Shown are a control neuron (left) and neurons treated for 15 minutes with the D1/D5-selective agonist SKF-38393 (right). The pseudocolor scale at left in the control image indicates GFP fluorescence levels. Scale bar=15 μm. B, Time-lapse imaging of a control neuron (top panel) shows a small decrease in GFP signal as seen in the ΔF/F plot for images before and 60 minutes after vehicle treatment. In contrast, a neuron treated with SKF for 15 minutes (bottom panel) shows an overall increase in GFP signal, with small hotspots of high-intensity fluorescence throughout the dendrite. Images of the dendrites before (top) and 60 minutes after vehicle or SKF treatment (bottom) are shown in the white box beneath each ΔF/F plot, which is aligned to the dendrite shown. C, Between-dish (A) summary data showing a significant increase in GFP fluorescence in the dendrites of SKF-treated neurons relative to control neurons (n=28 dendrites per condition, p<0.01). D, Time-lapse (B) summary data 60 minutes after agonist application showing a significant increase in GFP signal at distances greater than 75 microns from the cell soma (n=12 dendrites per condition, asterisk indicates p<0.05).

FIG. 11 A dopamine agonist stimulates the local translation of endogenous proteins as indicated by novel puromycin-based reporter of protein synthesis. A, A control neuron incubated for 15 minutes in F2P (left) exhibits moderate levels of fluorescence primarily due to basal rates of protein synthesis in the unstimulated cell. B, Neurons treated with the dopamine agonist SKF for 15 minutes in F2P show markedly higher fluorescence, with signal apparent throughout the dendritic arbor. The region boxed in yellow is shown at high power (right), where signal in the spines is clearly evident. Scale bars=20 (left), and 5 μm (right). C, A solution containing dihydrexidine (DHX), F2P, and the dye Alexa 568 (to mark solution flow) was perfused for 15 minutes onto a small dendritic segment of a cultured hippocampal pyramidal cell (left; shown is dye spot) resulting in a strong dendritic F2P signal (right). The high-power image (right, inset) shows high levels of F2P incorporation indicating local protein synthesis in the stimulated dendrite. D, Pretreatment and perfusion with a solution containing anisomycin abolished most of the DHX-induced F2P incorporation in the dendrite (compare high-power insets at right). E, The average F2P pixel intensity in each perfused region of interest (ROI, defined by the area of dendrite beneath the Alexa 568 dye) is shown as a series of box plots (see methods for a description of box plots). Perfusion of dendrites with DHX resulted in significantly greater F2P incorporation when compared to control dendrites (p<0.05). The enhancement produced by DHX was completely blocked by preincubation and perfusion with anisomycin (p<0.01, n=8 dendrites for each condition).

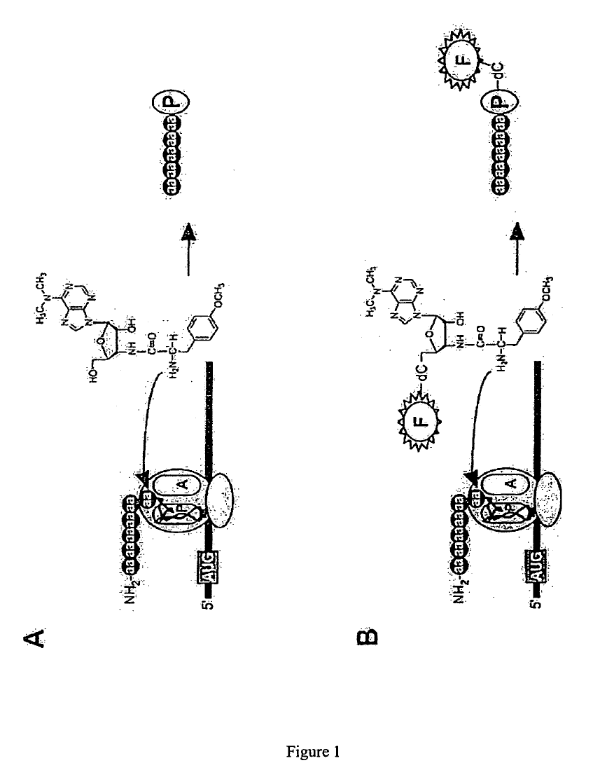

### DETAILED DESCRIPTION OF THE INVENTION

Before the present compositions and methods are described, it is understood that this invention is not limited to the particular methodology, protocols, and reagents described as these may vary. It is also to be understood that the terminology used herein is for the purpose of describing particular embodiments only, and is not intended to limit the scope of the present invention which will be described by the appended claims.

It must be noted that as used herein and in the appended claims, the singular forms “a”, “an”, and “the” include plural reference unless the context clearly dictates otherwise. Thus, for example, reference to “a cell” includes a plurality of such cells, reference to “a protein” includes one or more proteins and equivalents thereof known to those skilled in the art, and so forth.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the methods, devices, and materials are now described. All publications mentioned herein are incorporated herein by reference for the purpose of describing and disclosing the proteins, compounds, and methodologies which are reported in the publications which might be used in connection with the invention. Nothing herein is to be construed as an admission that the invention is not entitled to antedate such disclosure by virtue of prior invention.

The conjugate of the present invention comprises a “label moiety” that comprises a label substance and a moiety having an ability of binding to the C-terminal of a protein through a translation apparatus. The label moiety and binding moiety are linked through a chemical bond. The label moiety and binding moiety may be chemically bound either directly or thorough a linker.

The label moiety may be, but is not limited to, a non-radioactive label substance. The non-radioactive label substance includes fluorescent substances, coenzymes such as biotin, proteins, peptides, sugars, lipids, dyes, polyethylene glycol, and the like. The kind and size of the compounds are not limited unless the binding of the conjugate to the C-terminal of protein is prevented.

The fluorescent substance may be any type of fluorescent dye as far as it has a free functional group (for example, a carboxyl group, a hydroxyl group, an amino group, etc.) and can be bound to the binding moiety through a linker (for example, fluorescein series, rhodamine series, eosin series, NBD series, etc.). In one embodiment, the fluorescent substance is one belonging to the fluorescein series (see, e.g., Haugland, R. 1996. Handbook of fluorescent probes and research chemicals, 6th ed. Molecular Probes, Inc., Eugene, Oreg.) or the Cy series (e.g., Cy3 or Cy5).

The label moiety includes, from the viewpoint that a measuring apparatus is commercially applied, a radioactive substance or a fluorescent substance.

The binding moiety may be any compound as far as the compound has an ability of binding to the C-terminal of a synthesized protein when synthesis (translation) of the protein is carried out in a cell-free protein synthesis system or in a living cell. Usually, the binding moiety is a compound in which a compound containing a chemical structure skeleton that resembles a nucleic acid or its repeated structure and an amino acid or a compound having a chemical structure skeleton that resembles an amino acid are chemically bound to each other (nucleic acid derivative). There can be utilized those having an amido linkage as the chemical bond such as puromycin.

Also, there can be used those compounds in which a nucleoside or nucleotide and an amino acid are bound through an ester linkage (e.g., phosphodiester linkage). In addition, there can be utilized all the compounds that contain a linkage of any type allowing a compound having a chemical structure skeleton that resembles a nucleic acid and an amino acid or a compound having a chemical structure skeleton that resembles an amino acid to chemically bind to each other.

The term “phosphonate-puromycin” (including its derivatives) herein means a puromycin comprising a 5′-phophonate moiety in place of a 5′-phosphate moiety.

The term “puromycin-aminonucleoside” (including its derivatives) herein means a puromycin derivative that lacks an amino acid group on the amine sugar ring. In one aspect, the use of purine aminonucleoside (3′-amino-3′deoxy-N,N-dimethyl-adenosine) allows for facile substitution of amino acids and amino acid analogs for methoxyphenylalanine (e.g., phenylalanine and 4-methylphenylalanine). (See, e.g., Nguyen-Trung et al., J Org Chem (2003) 68:2038-2041).

The term “amino acid analog” means an amino acid that is naturally or non-naturally occurring and cannot be coded for by nucleic acids (e.g., glufosinate, gamma-hydroxyaspartate, omithine, 4-methylphenylalanine, etc.).

The term “nucleic acid” used herein means a nucleoside or its derivatives, or a repeated structure linked through a diester linkage with intervening phosphate between 3′-carbon and 5′-carbon.

The binding moiety comprises, but is not limited to comprise, a compound that comprises a nucleic acid and an amino acid or its derivative which are linked to each other. In one embodiment, the binding moiety comprises a compound that comprises 2′- or 3′-aminoadenosine or its derivative and an amino acid or its derivative which are linked to each other. In a related aspect, the binding moiety comprises puromycin, phosphonate-puromycin, puromycin-aminonucleoside, and derivatives thereof.

Examples of the binding moiety include ribocytidyl puromycin, deoxycytidyl puromycin, and deoxyuridyl puromycin.

The ability of the conjugate which constitutes the binding moiety to bind to the C-terminal of a protein when the synthesis (translation) of the protein is carried out in a cell-free protein synthesis system or in a living cell can be evaluated by carrying out the synthesis of a protein in the cell-free protein synthesis system or in the living cell in the presence of that compound and measuring the production of a peptidyl compound.

The cell-free protein synthesis system or the living cell is not limited to a particular one as long as protein synthesis can proceed when a nucleic acid encoding the protein is added or introduced therein. As a cell-free protein synthesis system, such a system may be derived from procaryotic or eukaryotic cells, for example, cell-free protein synthesis systems of E. coli, rabbit reticulocyte, wheat germ and the like. In a related aspect, the protein synthesis system may be used either a cell-free transcription-translation system or a cell-free translation system depending on whether the nucleic acid used as a template is DNA or RNA.

The conjugate can be produced by linking the label moiety and the binding moiety by a known chemical linking method.

As an example in which the label portion comprises a non-radioactive substance, first puromycin and rC-β-amidite are coupled and then the protective group is removed to synthesize rCpPur. In a similar manner, dCpPur and dUpPur can be synthesized.

Also, fluorescent labeling compounds, for example, Fluorpur, in which a fluorescent dye, for example, fluorescein, as the label moiety and a compound comprising a nucleic acid bound to an amino acid or a compound having a chemical structure skeleton resembling an amino acid, for example, puromycin as the binding moiety are linked to each other through a chemical bond, can be obtained by coupling puromycin and fluoredite and then removing the protective group.

The protein to which the labeling compound is added at the C-terminal thereof is not limited to a particular one.

The protein to which the conjugate is added at the C-terminal thereof can be produced by the production method of the present invention described hereinbelow.

The production method for the above-described protein according to the present invention comprises the step of carrying out synthesis of a protein in a cell-free protein synthesis system or in a living cell in the presence of a conjugate comprising a label moiety comprising a label substance and a binding moiety comprising a compound having an ability of binding to a C-terminal of a synthesized protein when protein synthesis is carried out in the cell-free protein synthesis system or in the living cell, the conjugate being present at a concentration effective for the labeling compound to bind to the C-terminal of the synthesized protein via peptide bond formation.

As described above, the compound that constitutes the binding moiety of the conjugate has an ability of binding to a C-terminal of a synthesized protein when protein synthesis is carried out in a cell-free protein synthesis system or in a living cell so that the labeling compound could inhibit protein synthesis in a concentration dependent manner.

For example, puromycin is known to inhibit the protein synthesis of bacteria (Nathans, D. (1964) Proc. Natl. Acad. Sci. USA, 51, 585-592; Takeda, Y. et al. (1960) J. Biochem., 48, 169-177) and animal cells (Ferguson, J. J. (1962) Biochim. Biophys. Acta, 57, 616-617; Nemeth, A. M. & de la Haba, G. L. (1962) J. Biol. Chem., 237, 1190-1193). The chemical structure of puromycin resembles that of aminoacyl tRNA and reacts with peptidyl tRNA that is bound to the P-site of ribosome and liberated from the ribosome as peptidyl puromycin, resulting in termination of the protein synthesis (Harris, R. J. (1971) Biochim. Biophys. Acta, 240, 244-262).

However, in the present invention, the protein synthesis is carried out in the presence of the conjugate at a concentration and under conditions effective for the conjugate to bind to the C-terminal of the synthesized protein, that is, at a concentration and under conditions where the protein synthesis in a cell-free protein synthesis system or in a living cell is not inhibited and where it can be linked in an amount allowing detection of the protein via linking the conjugate to the C-terminal of the protein.

Though not desiring to be bound to any theory, the conjugate is linked to the C-terminal of the synthesized protein when a termination codon comes to the A-site of a ribosome and the conjugate is linked to the C-terminal of protein by the action of peptidyltransferase in competition with the termination factor.

The concentration which is effective for the labeling compound to bind to the C-terminal of the synthesized protein can be determined by the method described in the examples below.

Unless otherwise indicated, gene manipulating techniques such as construction of plasmids, translation in a cell-free protein synthesis system or the like can be operated by the method described in Sambrook et al. (1989) Molecular Cloning, 2nd Edition, Cold Spring Harbor Laboratory Press or a method similar thereto.

According to the present invention, it is possible to label the C-terminal of a protein synthesized by translation using a cell-free protein synthesis system or a living cell regardless of whether it is from a procaryote or an eucaryote and therefore, the identification and function analysis of a protein expressed by the gene can be practiced rapidly, accurately, and economically.

Existing methods to study in vivo protein synthesis generally require choice of a candidate gene, radioactivity, or the destruction of cells. To overcome these limitations, a new class of reagents is disclosed that enables detection of protein synthesis in live cells using fluorescent and biotinylated puromycin conjugates. These reagents, of the general form X-dC-puromycin, are active in vitro and in vivo and provide a non-toxic alternative for the study of protein synthesis in live cells. A wide variety of label moieties appear to be accommodated at the X-position allowing for facile custom reagent design and development. Initial in vitro studies correlate the function of the disclosed compounds in peptide bond formation during protein synthesis. Subsequent in vivo experiments in a mouse thymocyte cell line demonstrate the usefulness of these molecules as indicators of protein synthesis in live cells. Selective enrichment studies with several conjugates as well as Western analysis demonstrate that these compounds all label protein in cells by the same general mechanism, attachment to nascent proteins during translation. The present results thus provide evidence that puromycin conjugates may serve as an alternative to existing tools to elucidate the proteome.

In one embodiment, a technique is disclosed to detect protein synthesis in live cells that does not require gene transfection or radiolabeling. The strategy thus provides an important potential alternative to these methods for studying protein expression in vivo. Generally, a great diversity of reagents of the class X-dC-puromycin, where X can be one or two fluorescent or affinity tags, can be constructed and show good activity in protein synthesis in vitro and in vivo. These reagents all appear to act by the same basic mechanism, entering the ribosomal peptidyl transferase site during translation, followed by covalent attachment to proteins being actively synthesized. Ribosome entry and attachment occurs predominantly at a few discrete sites in the open reading frame including the stop codon, rather than at every position in the chain [14,25]. Previous work also demonstrates that over a 50-fold concentration range that brackets the IC₅₀, the length of truncated products is the same and that shorter products are favored as the conjugate concentration is increased substantially.

Despite the intermediate size of these molecules (1163 to 1730 Da), all the conjugates appear to be competent to enter the D9 suspension tissue culture cells as used here and act at modest concentrations (5-25 μM). Experiments with other mammalian and insect cell types support the idea that the ability of these compounds to cross membranes and act in protein synthesis is a general phenomenon (W. B. Smith, E. Schuman, B. Hay, unpublished observations).

All of the conjugates examined show a significant and measurable shift in the fluorescence intensity of live cells as compared to the control conjugates. Western analysis and selective enrichment studies support the idea that this shift is due to the specific covalent attachment of the conjugates to nascent proteins during translation. Demonstration that affinity tags may be inserted into expressed proteins in vivo provides one of skill in the art the ability to examine protein expression in response to various cellular stimuli and subsequent identification of the individual polypeptides through a combination, for example, of affinity purification and mass spectrometry-based sequence analysis.

These compounds are relatively non-toxic based when used for short duration (approximately 24 hrs) on the proportion of live cells seen in flow cytometry experiments. The robust labeling and signal to noise observed thus makes these compounds useful for a great diversity of cell, tissue, and organism-level experiments. The long-term toxicity of the present set of compounds may provide some limitations for their use. In one aspect, non-toxic variants that can be photoactivated or presented as pro-drugs are envisaged. The general class of compounds described herein should therefore serve as useful cell biology tools to evaluate in vivo protein synthesis in areas such as nuclear protein synthesis [26, 27], neuron dentritic protein synthesis [10], dendritic cell aggresome-like induced structures (DALIS) [28], and other novel proteome functions.

The following examples are intended to illustrate but not limit the invention.

### EXAMPLES

### Example 1

### Experimental Procedures/Materials

L-Puromycin hydrochloride, rabbit globin mRNA, and carboxypeptidase Y (CPY) were obtained from Sigma Chemical Co. (St. Louis, Mo.). Rabbit reticulocyte Red Nova® lysate was purchased from Novagen (Madison, Wis.). L-[³⁵S]methionine ([³⁵S]Met) (1175 Ci/mmol) was obtained from NEN Life Science Products (Boston, Mass.). Immunopure® immobilized Neutravidin-agarose was from Pierce (Rockford, Ill.). GF/A glass microfiber filters were from Whatman.

Puromycin Conjugates

Puromycin conjugates were synthesized using standard phosphoramidite chemistry at the California Institute of Technology oligonucleotide synthesis facility. Puromycin-CPG was obtained from Glen Research (Sterling, Va.). Oligonucleotides were synthesized with the 5′-trityl intact, desalted via OPC cartridge chromatography (Glen Research) (DNA oligonucleotides only), cleaved, and evaporated to dryness. 5′-Biotin phosphoramidite, Biotin phosphoramidite, 5′-Fluorescein phosphoramidite, 6-Fluorescein phosphoramidite (Glen Research) were used to make the biotin- and dye-puromycin conjugates. Ac-dC-Me-phosphonamidite (Glen Research) was used to prepare the phosphonate puromycin conjugates. The dried samples were resuspended and desalted on Sephadex G-25 (Sigma). Puromycin, puromycin-conjugate, and control molecule concentrations were determined with the following extinction coefficients (M-1 cm-1): puromycin (ε260=11,790; in H20); B2P and B2P-Me (ε260=19,100; in H20); F2P, F2P-Me, DMT-F2P-Me, FB2P, BF2P, F2A, and BF2A (ε471=66,000; in 1× PBS); Cy52P and Cy52A (ε650=250,000; in 1× PBS).

In Vitro Potency Determination for Puromycin Conjugates

Translation reactions containing [³⁵S]Met were mixed in batch on ice and added in aliquots to microcentrifuge tubes containing an appropriate amount of puromycin conjugate (or control molecule) dried in vacuo. Typically, a 20 μl translation mixture consisted of 0.8 μL of 2.5 M KCl, 0.4 μL of 25 mM MgOAc, 1.6 μL of 12.5× translation mixture without methionine, (25 mM dithiothreitol (DTT), 250 mM HEPES (pH 7.6), 100 mM creatine phosphate, and 312.5 μM of 19 amino acids, except methionine), 3.6 μL of nuclease-free water, 0.6 μL (6.1 μCi) of [³⁵S]Met (1175 Ci/mmol), 8 μL of Red Nova nuclease-treated lysate, and 5 μL of 0.05 μg/μL globin mRNA. Inhibitor, lysate preparation (including all components except template), and globin mRNA were mixed simultaneously and incubated at 30° C. for 60 min. Each reaction (2 μL) was combined with 8 μL of tricine loading buffer (80 mM Tris-Cl (pH 6.8), 200 mM DTT, 24% (v/v) glycerol, 8% sodium dodecyl sulfate (SDS), and 0.02% (w/v) Coomassie blue G-250), heated to 90° C. for 5 min, and applied entirely to a 4% stacking portion of a 15% tricine-SDS-polyacrylamide gel containing 20% (v/v) glycerol [29] (30 mA for 1 h, 30 min). Gels were fixed in 10% acetic acid (v/v) and 50% (v/v) methanol, dried, exposed overnight on a Phosphorlmager screen, and analyzed using a Storm Phosphorlmager (Molecular Dynamics).

Neutravidin Capture of In Vitro Translated Protein-Puromycin-Conjugate Products

Neutravidin-agarose [50% slurry (v/v)] was washed 3 times with 1× PBS+0.1% Tween-20 and resuspended in 1 mL of 1× PBS+0.1% Tween-20. To 200 μL of this suspension, 12 μL of the reaction lysate and 0.8 mL of 1× PBS+Tween-20 were added. The samples were rotated at 4° C. for 3 h and washed with 1× PBS+Tween-20 until the cpm of [³⁵S]Met were <500 in the wash. The amount of immobilized [³⁵S]Met-protein-puromycin conjugate was determined by scintillation counting of the Neutravidin-agarose beads.

Preparation of MIGPAC Infected 16610D9 Cells

The PAC gene was cloned into MIG using BgII and EcoRI restriction sites to yield MIGPAC. 293T-HEK fibroblasts (American Tissue Culture Collection) were co-transfected with pECL-Eco [30] and MIG or MIGPAC by calcium phosphate precipitation. After 12 hours, the precipitate was removed, cells were washed once with PBS, and 4 mL of fresh complete Dulbecco's Modified Eagle Medium (DMEM) supplemented with 10% fetal calf serum (FCS). Viral supernatant was removed 24 hours later and used in infection of 16610D9 cells. One million D9 cells were spin-infected with 0.4 mL of viral supernatant suplemented with 5 μg/ml Polybrene (Sigma-Aldrich).

Enrichment of GFP(+)16610D9 Cells Using Puromycin and Puromycin Conjugates

16610D9 cells infected with either MIG of MIGPAC were cultured in RPMI media with 10% FBS and grown at 3 ° C. in a humidified atmosphere with 5% CO₂. For each experiment, 16610D9 cells (0.25×10⁶/well) were added to 24-well microtiter plates along with puromycin, puromycin-conjugate, and control molecules dissolved in the minimum amount of either media or PBS. After a 48 h incubation, the cells were washed twice in 2 mL PBS+4% FCS and resuspended in PBS+4% FCS supplemented with 2% formaldehyde along with incubation at 37° C. for 10 min. Flow cytometry was carried out on a Beckman FACScabilur Flow Cytometer.

Detection of Protein Synthesis Events In vivo using Flow Cytometry

16610D9 cells (0.5 million mL⁻¹) were combined with the various puromycin conjugates and control molecules resuspended in the minimum volume of PBS or media as described above. After a 24 h incubation, the cells were washed twice in 2 mL PBS+4% FCS and resuspended in PBS+4% FCS supplemented with 2% formaldehyde followed by incubation at 37° C. for 10 min or used directly after washing for immediate flow cytometry analysis.

Western Analysis of 16610D9 Cells Treated with Puromycin Conjugates

Cells were prepared as described above except as indicated anisomycin was added to a final concentration of 250 μg mL⁻¹ and washed twice in PBS. Live cell number was determined using trypan blue exclusion dye and each sample was adjusted to contain an equal number of live cells. Cell pellets were resuspended in 2× lysis buffer (100 mM β-glycerophosphate, 3 mM EGTA, 2 mM EDTA, 0.2 mM sodium-orthovanadate, 2 mM DTT, 20 μg/ml aprotinin, 20 μg/ml leupeptin, 50 μg/ml trypsin inhibitor, and 4 μg/ml pepstatin, and 1% Triton X-100) and incubated on ice for 30 min. Cell debris was removed by centrifugation at 20,000×g for 30 min. Cell lysate was combined with SDS loading buffer (0.12 M Tris-Cl (pH 6.8), 20% glycerol, 4% (w/v) SDS, 2% (v/v) β-mercaptoethanol, and 0.001% bromophenol blue) and heated at 90° C. for 10 min. Samples were applied entirely to a 4% stacking portion of a 10% glycine-SDS-polyacrylamide gel (30 mA for 1 h, 30 min). Protein was transferred using standard Western transfer techniques and the blot was probed with an anti-fluorescein antibody followed by an anti-rabbit-horseradish peroxidase conjugate (Pierce chemicals). The chemiluminescence reaction was carried out using the ECL PLUS Western Blotting Direction System (Amersham BioSciences).

### Example 2

### Design of Puromycin Conjugates

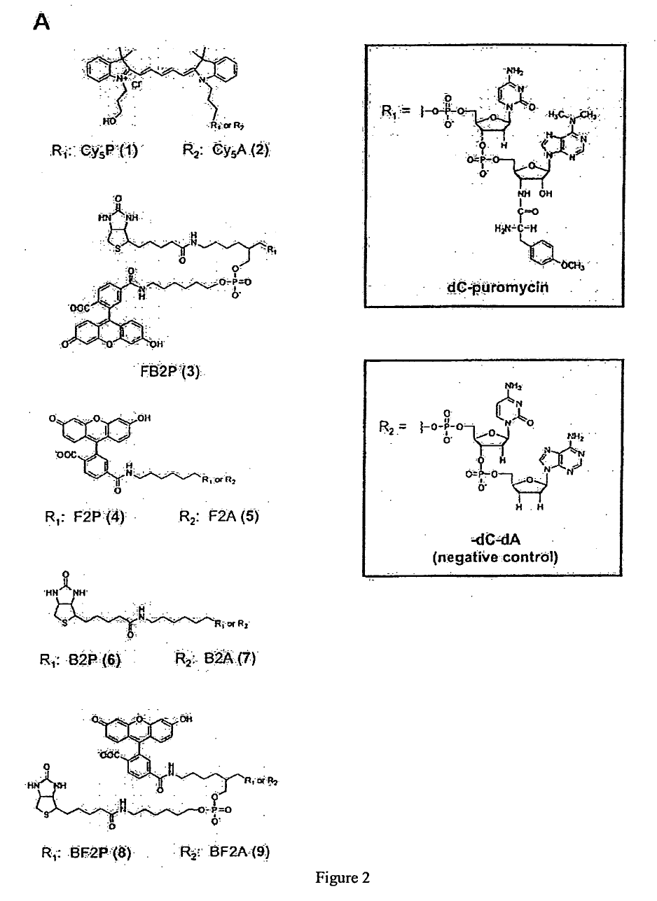

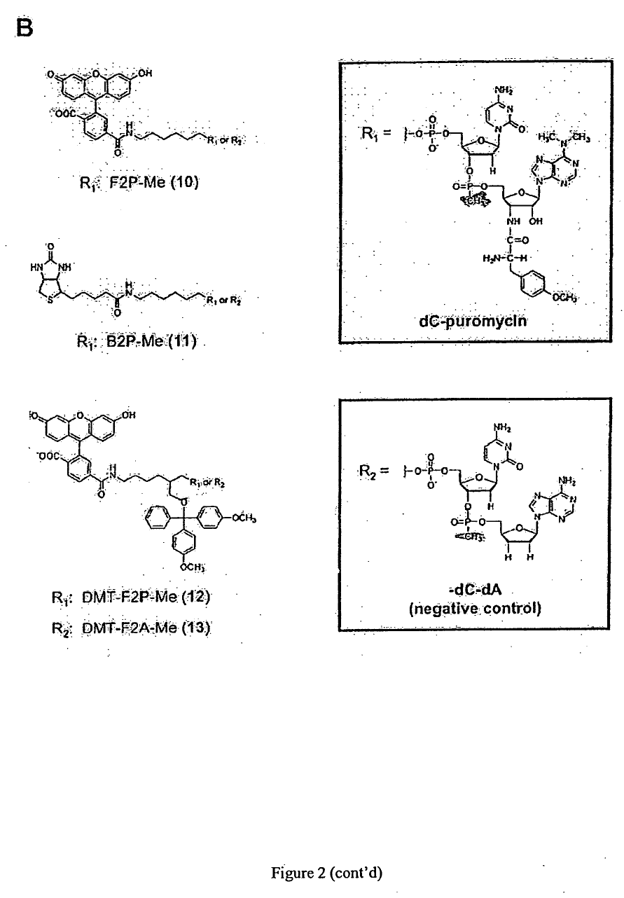

To label newly synthesized proteins, puromycin conjugates would have to satisfy three general criteria: 1) functionality in peptide bond formation, 2) cell permeability, and 3) ready detection in a cellular or biochemical context. In addressing the first issue, it had been previously shown that puromycin derivatives bearing substitutions directly off the 5′ OH functioned poorly in vitro (e.g., biotin-puromycin IC₅₀=54 μM) [14], whereas conjugates with the general form X-dC-puromycin (e.g., biotin-dC-puromycin) were substantially more effective (IC₅₀=11 μM) [14]. Therefore, a molecule design was determined by varying the substituents appended to dC-puromycin (FIG. 2A).

In order to facilitate cellular entry and detection, a number of factors were considered including: 1) type and position of the label, 2) the linker between the label and dC-puromycin, 3) background fluorescence properties, and 4) membrane permeability including net charge and hydrophobicity. Various dC-puromycin conjugates were designed and synthesized to address these issues systematically. The first series of puromycin conjugates (1, 2, 4, 6, 8; FIG. 2A) either contain fluorescent dyes (compounds 2 and 4), biotin (compound 6), or both (compounds 1 and 8). Two different fluorescent dyes were utilized (fluorescein and Cy5) to provide detection at a range of emissions. Biotin labels were introduced to enable detection via western blot analysis or affinity purification. A series of compounds ( 3, 5, 7, 9; FIG. 2A), which lack the 3′-amino acid moiety to serve as negative controls were also prepared.

A second series of conjugates with a phosphonate linkage between dC and puromycin were prepared to examine whether reduction of charge would enhance cell membrane solubility and facilitate cellular entry (FIG. 2B). Three compounds (10, 11, 12; FIG. 2B) were constructed bearing fluorescein (10, F2P-Me), biotin (11, B2P-Me), or the hydrophobic dimethoxytrityl group (DMT) and fluorescein (12, DMT-F2P-Me). A DMT bearing fluorescein-dC-dA conjugate (DMT-F2A-Me) served as a negative control (13; FIG. 2B). The DMT group was added to gauge whether the addition of a hydrophobic group would further enhance entry into cells.

### Example 3

### Analysis of Puromycin-Conjugate Activity In Vitro

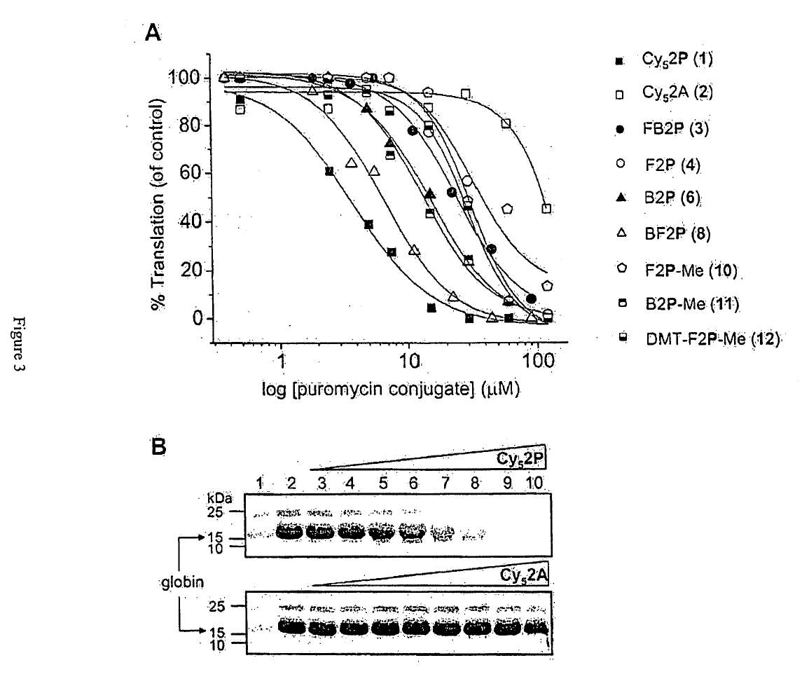

Initial analysis began by examining the activity of each of the conjugates in vitro for their ability to inhibit protein translation. Previously, this activity assay had been used to measure the IC₅₀ for various puromycin conjugates [14] and analogues [18], as well as demonstrate a direct relationship between the IC₅₀ and the efficiency of protein labeling [14]. Using this approach, IC₅₀ values were measured for the compounds in FIGS. 2A and 2B (FIG. 3A). High resolution SDS-tricine gel data corresponding to a typical IC₅₀ determination is shown for Cy52P (1) and Cy52A (2) (FIG. 3B). Generally, the activity of conjugates with the form X-dC-puromycin falls over a fairly narrow range in vitro, with IC₅₀ values ranging from ˜4 to ˜30 μM (Table 1). Also, control conjugates that lack the amino acid moiety, e.g., Cy52A (2) and BF2A (9), show little ability to inhibit protein synthesis even at high concentrations.

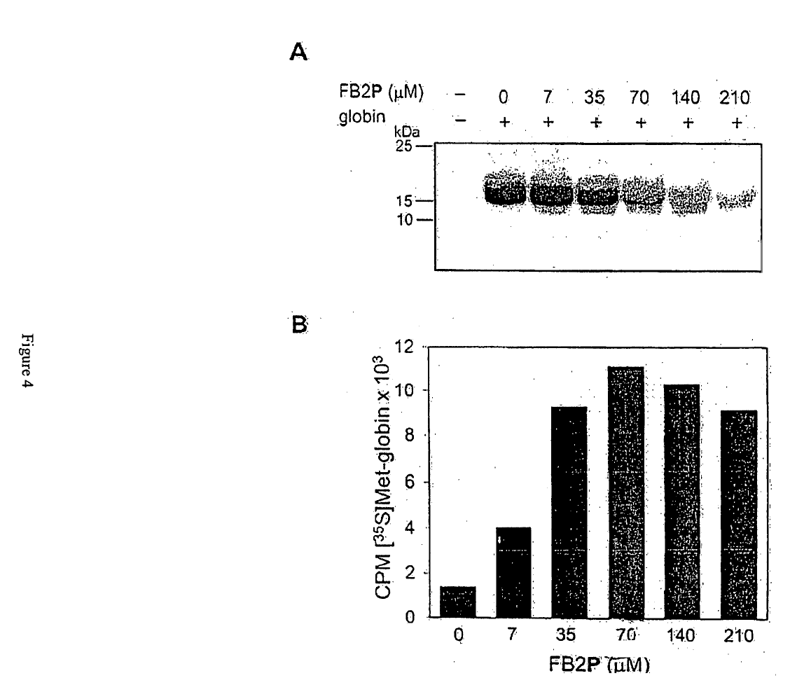

Confirmation that the puromycin conjugates could become covalently attached to protein in vitro was attempted next. To do this, globin mRNA was translated in the presence of increasing concentrations of FB2P (3), a conjugate containing fluorescein and biotin moieties (FIG. 4A). Next, the concentration-dependent incorporation of FB2P was analyzed using neutravidin affinity chromatography of these same translation reactions (FIG. 4B). These data indicate that puromycin conjugates are incorporated efficiently over a broad concentration range ranging from 2- to 3-fold below the IC₅₀ to well above it. Thus, labeling is possible even at concentrations where protein synthesis is not greatly inhibited.

These observations support the development of a broad range of puromycin-based reagents for two reasons. First, compounds of the form X-dC-puromycin appear tolerant to a wide variety of substitutions, including molecules containing more than one detection handle (e.g., BF2P and FB2P). Interestingly, even the methyl phosphonate versions (F2P-Me, 10; B2P-Me, 11; DMT-F2P-Me, 12) showed good levels of in vitro activity. Second, the IC₅₀ values indicate that even modest concentrations of each of these reagents in the low micromolar range will be sufficient to achieve good levels of protein labeling. This is because the instant data (Table 1, FIGS. 3, 4) as well as previous data [14,18], demonstrate that protein labeling is achieved at or below the IC₅₀ value.

TABLE 1
The concentration of puromycin conjugate required for 50% inhibition
of globin translation (IC₅₀).*

| Puromycin conjugate | IC₅₀ (μM) |
|---|---|
| (1) FB2P | 24 |
| (2) Cy₅2P | 3.8 |
| (3) Cy₅2A | >100 |
| (4) F2P | 22 |
| (10) F2P-Me | 25 |
| (11) DMT-F2P-Me | 29 |
| (8) BF2P | 5.8 |
| (6) B2P | 15 |
| (11) B2P-Me | 16 |

*In replicate experiments, the standard error is <5%.

Thus, these in vitro translation and protein labeling assays provided a starting concentration range for analysis in live cells.

### Example 4

### Analysis of Puromycin-Conjugate Activity In Vivo

In order to analyze the activity of puromycin conjugates in vivo, choosing of both an appropriate cell line and an appropriate quantitation and detection scheme was needed. While microscopy is a powerful means to analyze individual cells and small sections of tissue, performance of experiments where thousands to millions of cells could be examined for protein labeling was desired. Therefore, flow cytometry was chosen as the primary means to analyze uptake and incorporation of the conjugates. In addition to providing a quantitative measure of fluorescence and cell size, flow cytometry methods enable live cells and dead cells to be readily distinguished [19]. The mammalian thymocyte D9 cell line (16610D9) [20] was chosen for four reasons: 1) they have relatively uniform size and shape, 2) they do not aggregate, making single cell detection possible, 3) they are suspension cells, which allows for ready growth in culture with subsequent acquisition of a large number of single cell readings using flow cytometry, and 4) they are amenable to routine infection techniques to introduce selectable markers and GFP-based tags.

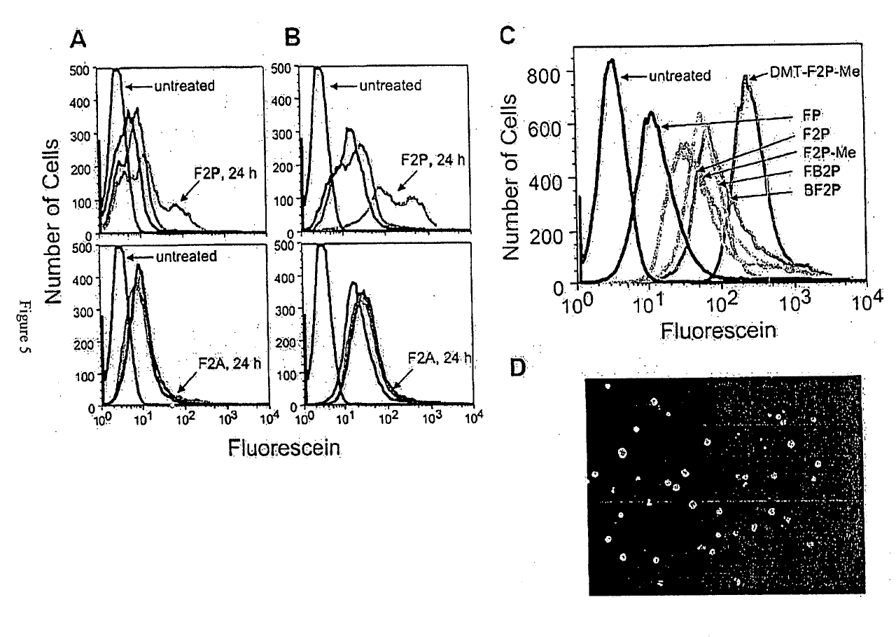

Comparing the concentration and time dependence of labeling with F2P (4) and the negative control conjugate F2A (5) (FIGS. 5A, B) was selected first. For F2P, progressively increased fluorescence is seen with increasing time (FIGS. 5A, B) and the greatest enhancement is seen after the 24 h incubation at both 5 μM and 25 μM of the conjugate. At both concentrations, a substantial population of live cells is detected and demonstrates up to 4-fold enhanced fluorescence relative to the F2A control molecule. Longer incubation (48 hours) in the presence of F2P eventually kills the majority of cells at both concentrations tested. In contrast, the background fluorescence from F2A reaches a maximum of ˜101 units after a 7 h incubation for both 5 and 25 μM incubations (FIGS. 5A, B) and F2A has no apparent effect on cell viability. The fluorescence enhancement beyond 101 units for cells treated with F2P is consistent with C-terminal protein labeling by the fluorescein-puromycin conjugate. These experiments also suggest that there is an optimum concentration and incubation time for labeling expressed proteins without killing the cells.

The relative level of fluorescence enhancement for a series of conjugates was selected next. To do this, a uniform population of D9 cells was split into separate containers, each containing identical concentrations of a different puromycin conjugate, incubated for 24 hours, and analyzed by flow cytometry with a live-cell gate as before (FIG. 5C). In this series, DMT-F2P-Me (12) gives the strongest enhancement and the rank order of compounds follows DMT-F2P-Me (12)>FB2P (1)˜BF2P (8)>F2P (4)˜F2P-Me (10)>FP. The IC₅₀ values for all the compounds with the exception of FP (IC₅₀=120 μM [14]) are relatively similar, while addition of the DMT group in compound (12) would be expected to confer increased hydrophobicity and membrane permeability. Compounds containing a phosphate (F2P (4)) or a methylphosphonate (F2P-Me (10)) bridging the puromycin and dC residue show little difference in IC₅₀ (FIG. 3, Table 1) and in vivo labeling (FIG. 5C), arguing that charge at this position does not play a key role in either the activity as a substrate or entry into the cell. The poor IC₅₀ for FP in vitro [14] correlates with the small fluorescence enhancement seen for this compound in vivo (FIG. 5C). Epi-fluorescence microscopy confirms that the conjugate DMT-F2P-Me (12) readily enters and labels D9 cells brightly (FIG. 5D).

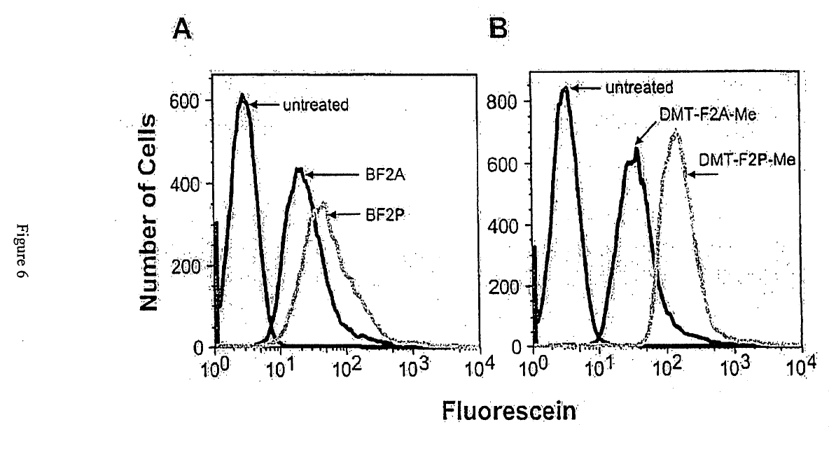

Following these experiments, confirmation that two of the best compounds, BF2P (8) and DMT-F2P-Me (12) also showed fluorescence enhancement in vivo relative to control molecules containing only a terminal adenosine was attempted. Indeed, comparison of cells treated with BF2P (8) versus BF2A (9) (FIG. 6A) and DMT-F2P-Me (12) versus DMT-F2A-Me (13) (FIG. 6B), indicates that compounds bearing the terminal puromycin moiety show a 3- to 4-fold fluorescence enhancement as compared with the control molecules. This shift in fluorescence is consistent with labeling protein during rounds of translation. Overall, the combination of the in vitro and in vivo observations is consistent with the notion that the overall fluorescence enhancement reflects both the efficacy and the cellular permeability of the compounds.

### Example 5

### Mechanism of Puromycin Conjugate Activity In Vivo

It was necessary to demonstrate that the puromycin conjugates as constructed were acting in vivo by the same mechanism as puromycin itself. Puromycin can be used as a selection agent in mammalian cell culture to kill cells that lack the resistance gene encoding puromycin N-acetyl-transferase (PAC) [21]. This enzyme N-acetylates the reactive amine on puromycin and blocks its ability to participate in peptide bond formation [22,23]. In a mixed population of cells, those that lack a vector expressing PAC can be selectively killed by long incubations (≧48 hours) with puromycin, leaving only vector-containing cells alive. Previously, it had been shown that chemical acylation inactivates puromycin-mediated translation inhibition in vitro [14]. Thus, it was desirable to demonstrate whether the D9 cells bearing PAC would be resistant to killing (and thus enriched in the mixed population) by long incubations with puromycin itself or the puromycin conjugates in vivo.

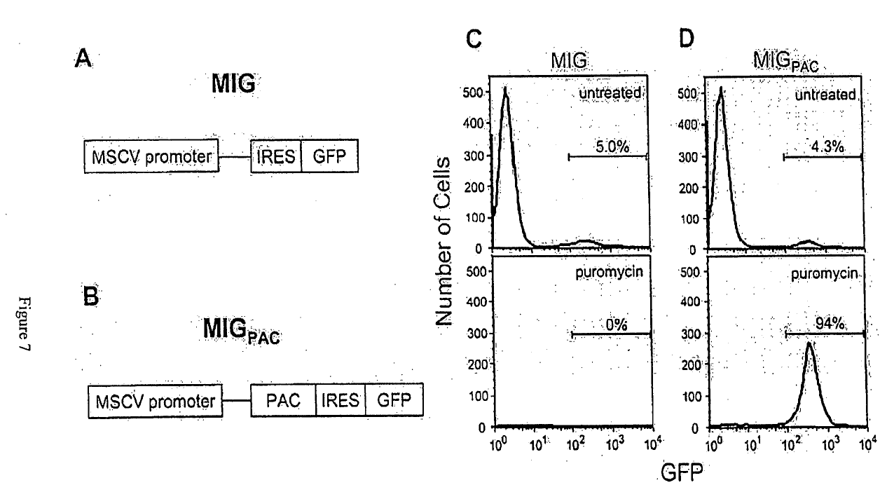

Foreign genes can be inserted into D9 cells by infection with a viral vector (see Experimental Procedures). Vectors that express GFP provide a straightforward means to measure the fraction of cells that become infected and a direct means to monitor any vector-mediated enrichment. Infected D9 cells were infected with a viral vector driven by a mouse stem cell virus promoter (MSCV) containing an internal ribosome entry site (IRES) upstream from enhanced green fluorescent protein (EGFP) referred to as MIG (MIG=MSCV-IRES-GFP; FIG. 7A) [24]. MIG expresses GFP so that infection efficiency can be monitored by GFP fluorescence (FIG. 7A). A second vector containing the PAC gene was also constructed (MIGPAC; FIG. 7B) and results in a bicistronic mRNA in which both PAC and GFP can be translated (FIG. 7B).

Flow cytometry was used to examine both the infection efficiency and confirm the ability to perform puromycin-based enrichment. After infection with the MIG or MIGPAC vectors, 5.0% and 4.3% of the D9 cells were infected and alive based on GFP expression, respectively (FIGS. 7C, D, upper panels). In both cases, the other 95% of the cells showed no GFP-based signal. Puromycin was then added to both MIG and MIGPAC infected cells followed by incubation for 48 h at 37° C. For MIG infected cells, puromycin results in almost complete killing of both GFP-positive and GFP-negative cells (FIG. 7C, lower panel). For MIGPAC infected cells, puromycin selectively kills only those cells lacking GFP, such that after 48 hours the population is totally dominated by GFP-positive cells (94%) (FIG. 7D, lower panel). Enrichment of GFP-positive cells occurs because they express the PAC resistance protein that acylates puromycin, rendering it inactive. These experiments demonstrate that puromycin acylation is sufficient to rescue cells from puromycin toxicity and that N-blocked puromycin is non-toxic to D9 cells. The selective enrichment of PAC-expressing cells argues that puromycin exerts its effect on D9 cells by acting on the translation apparatus in vivo.

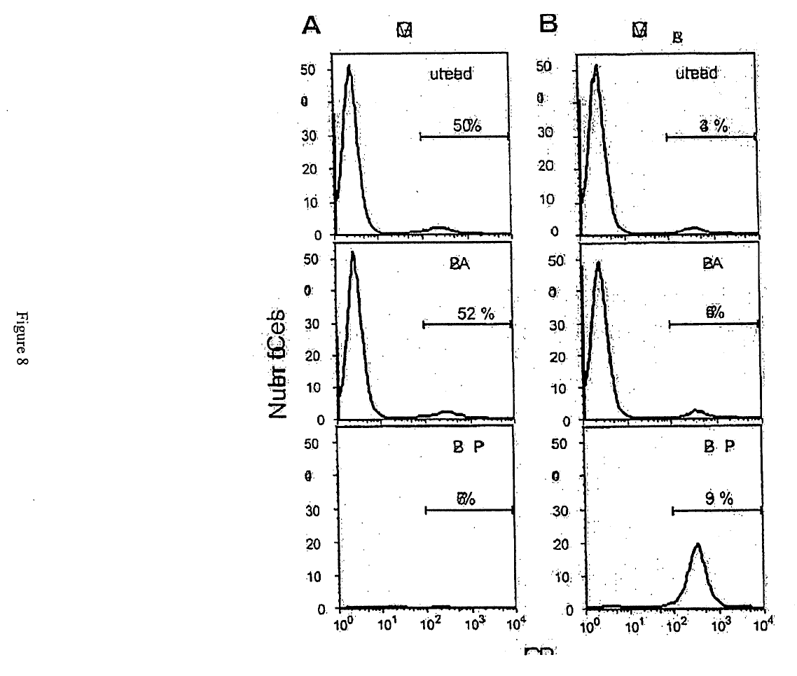

B2P (6) was examined to determine whether it could act in a biochemically similar fashion as puromycin itself. As with puromycin, flow cytometry indicated that long exposures of B2P (6) kills the vast majority of the cells infected with MIG (FIG. 8A bottom panel), while B2A (7), a control molecule lacking the amino acid, had no effect (FIG. 8A, middle panel). Importantly, cells infected with MIGPAC show selective enrichment when incubated with B2P (6) (FIG. 8B, bottom panel), while B2A shows no change in GFP-positive and negative populations (FIG. 8B, middle panel). These experiments are fully consistent with B2P (6) acting by the same mechanism as puromycin itself. Further, these data also provide the first demonstration that PAC can act on puromycin conjugates bearing 5′-extensions in vivo.

In line with this conclusion, two other puromycin conjugates show similar activity with B2P. Cy5-bearing conjugate Cy52P (2) was examined and compared its action with an analogous control molecule, Cy52A (3), using both MIG and MIGPAC infected cells. Cy5 provides a useful spectroscopic handle in this context because its red-shifted fluorescence allows the emission of the conjugate to be unambiguously separated from that of GFP. As with B2P versus B2A, MIG-infected cells were insensitive to Cy52A, while long exposure of Cy52P killed both GFP-positive and negative populations, since they lacked the PAC resistance determinant. Cy52P also selectively enriched MIGPAC infected cells from 4.3% to 90%. Additionally, B2P-Me (11) also resulted in selective enrichment of MIGPAC-bearing cells and had similar potency with B2P (6). Taken together, these data support the idea that the various X-dC-puromycin conjugates act by the same mechanism as puromycin in vivo and that conjugates lacking the 3′-amino acid moiety have no effect.

### Example 6

### Western Blot Analysis of Puromycin Conjugate Labeling in Live Cells

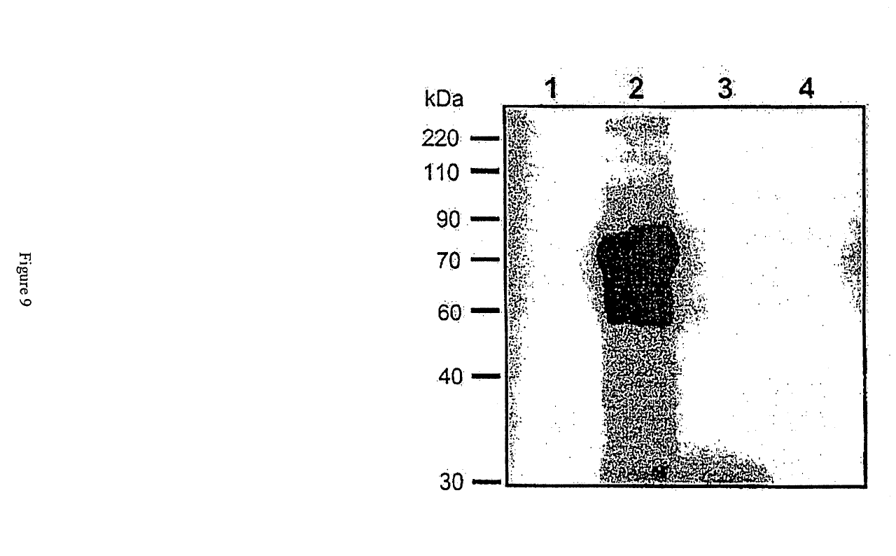

Action of puromycin and the conjugates should result in proteins bearing these compounds at their C-terminus in vivo. Western blot analysis of cellular lysates was chosen to examine if incorporation occurred in vivo and compare the resulting signal with the control conjugates. Cells were incubated with either BF2P (8) or the control molecule BF2A (9), washed, and a whole-cell lysate was prepared for each sample (see Experimental Procedures). Proteins were run on a SDS-PAGE gel and transferred to nitrocellulose. Equal protein loading was confirmed in each lane using Ponceau S. The Ponceau S stain was rinsed away and the blot was probed with an anti-fluorescein antibody to detect any fluorescein-conjugated protein containing BF2P or BF2A. Cells treated with BF2P (FIG. 9, lane 2) show good levels of incorporation in this assay, while lanes with cells alone (lane 1), cells treated with BF2A (lane 3), or anisomycin (lane 4) show essentially no signal (FIG. 9). The Western-blot analysis of BF2P thus shows good correlation with flow cytometry data and is consistent with a model where puromycin conjugates are stably incorporated into proteins in vivo during protein synthesis.

### Example 7

### Evaluation of Protein Expression in Hippocampal Neurons

The use-dependent modification of synapses is strongly influenced by the actions of the neuromodulator dopamine, a transmitter that participates in both the physiology and pathophysiology of animal behavior. In the hippocampus, dopaminergic signaling acting via the cAMP-PKA pathway is thought to play a key role in protein synthesis-dependent forms of synaptic plasticity [31-33]. The molecular mechanisms by which dopamine influences synaptic function, however, are not well understood. Using a green fluorescent protein (GFP)-based reporter of translation, as well as a novel, small-molecule reporter of endogenous protein synthesis, it was shown that dopamine D1/D5 receptor activation stimulates local protein synthesis in the dendrites of cultured hippocampal neurons. Furthermore, the GluRl subunit of AMPA receptors was identified s one protein upregulated by dopamine receptor activation. In addition to enhancing GluRl synthesis, dopamine receptor agonists increase the incorporation of surface GluRl at synaptic sites. The insertion of new GluRs is accompanied by an increase in the frequency, but not the amplitude, of miniature synaptic events. Together, these data suggest a local protein synthesis-dependent activation of previously silent synapses as a result of dopamine receptor stimulation.

Methods

Cultured Hippocampal Neurons

Dissociated hippocampal neurons were prepared and maintained as previously described [34]. Briefly, hippocampi from postnatal day 2 Sprague-Dawley rat pups were enzymatically and mechanically dissociated and plated into poly-lysine coated glass-bottom petri dishes (Mattek). Neurons were maintained for 14-21 days at 37° C. in growth medium (Neurobasal A supplemented with B27 and Glutamax-1, Invitrogen).

All images were acquired with an Olympus IX-70 confocal laser scanning microscope running Fluoview software (Olympus America, Inc). GFP, Alexa 488, and F2P were excited with the 488 nm line of an argon ion laser, and emitted light was collected between 510 and 550 nm. Alexa 568 was excited with the 568 nm line of a krypton ion laser, and emitted light was collected above 600 nm. In experiments where two channels were acquired simultaneously, settings were chosen to ensure no signal bleed-through between channels. For between-dish comparisons on a given day, all images were acquired at the same settings, without knowledge of the experimental condition during image acquisition. All post-acquisition processing and analysis was carried out with ImageJ (NIH) and Matlab (The MathWorks, Inc.). To facilitate the analysis of fluorescence signal as a function of distance from the soma, dendrites were linearized and extracted from the full-frame image using a modified version of the Straighten plugin for ImageJ.

Dendrites were analyzed for time-lapse as follows: fluorescence was averaged across the width of linearized dendrites, generating a vector of mean pixel intensities equal to the length of the dendrite, ΔF/F (Ftn-Ft0/Ft0) was then computed at each pixel along the dendritic length. A value of one was added to every pixel in the linearized dendrite image, to a maximum of 255, which sets the minimum mean pixel intensity across the width of the dendrite equal to one. This prevents artificially large ΔF/F values that result from fractional mean pixel values due to zeros in the initial image. For time-lapse summary data, the sum of ΔF/F values in 75-micron bins was computed for each dendrite, and the mean±standard error for all dendrites in a given experimental condition was plotted. 3D colocalization and particle analysis was performed using custom-written functions in Matlab. Of particular concern in such measurements is the issue of selecting appropriate threshold values to isolate the punctate data of interest from background noise in the raw images. In order to avoid potential biases in selecting thresholds, a “graythresh” command in Matlab was used. This function generates an optimal threshold based on Ostu's method, which sets a threshold that minimizes the intraclass variance of the black and white pixels. To further ensure that the experimental effects observed were robust to threshold settings, the colocalization and particle analysis was performed with a series of 7 to 11 thresholds, using the output of graythresh as the median threshold value. All reported results were unaffected by such a range of threshold settings.

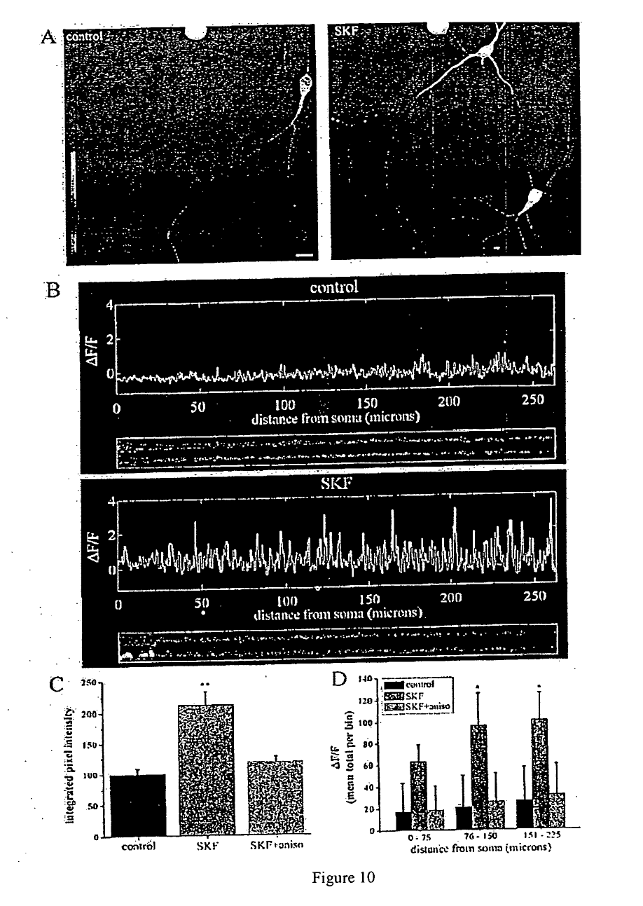

In initial experiments, the ability of a dopamine D1/D5 receptor agonist (SKF-38393) to stimulate protein synthesis by visualizing a GFP protein synthesis reporter molecule [34] in cultured hippocampal neurons was examined. The levels of GFP signal in control (untreated) neurons to neurons that had been exposed to bath application of the dopamine agonist was compared. Relative to controls, neurons treated with SKF (100 μM for 15 min) showed significantly enhanced protein synthesis in both the soma and dendrites (FIGS. 10A, C). Similar results were obtained with a different D1/D5 receptor agonist, dihydrexidine (DHX). The stimulation of protein synthesis by SKF was completely prevented by the co-application of a D1/D5 receptor antagonist (SCH-23390; 10 μM), confirming that the observed effects are due to dopamine receptor activation [mean percent inhibition of SKF-stimulated protein synthesis: 97.3±5.1%; n=12]. The time course of SKF-induced protein synthesis was next examined using time-lapse imaging of dendrites. Control dendrites exhibited relatively stable levels of GFP fluorescence over a 60 minute imaging period (FIG. 10B). In contrast, a brief (15 min) exposure to SKF increased the GFP signal in dendrites within 60 minutes (FIGS. 10B, D). In both sets of experiments, the effects of SKF were completely prevented by co-application of the protein synthesis inhibitor anisomycin (FIGS. 10C, D), indicating that D1/D5 receptor activation stimulates protein synthesis in hippocampal neurons.

The above data show that dopamine agonists can stimulate the synthesis of a fluorescent protein synthesis reporter that contains the 5′ and 3′ untranslated regions from α-CaMKII [34].

### Example 8

### Translation of Endogenous mRNA

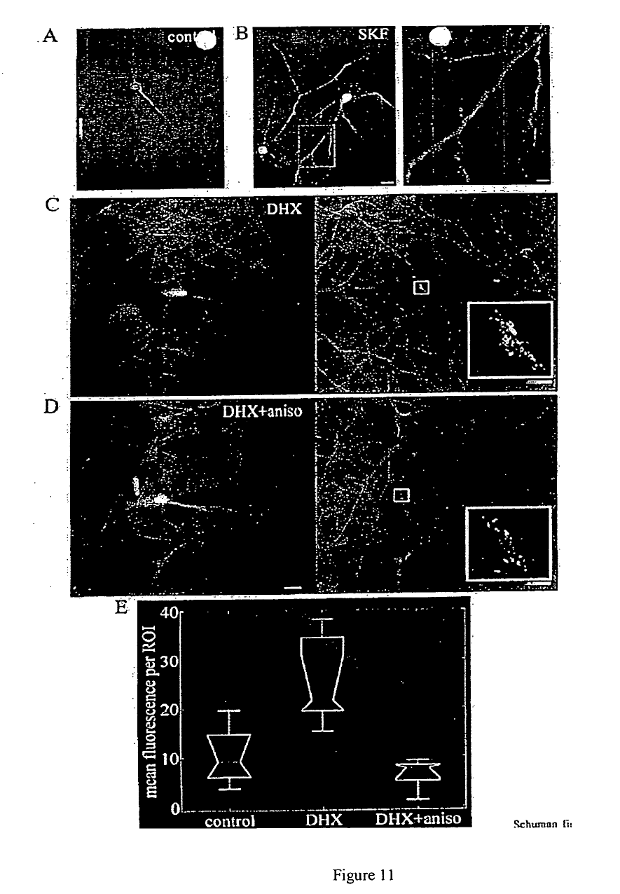

To determine whether D1/D5 receptors activate the translation of endogenous mRNAs in living neurons, fluorescein-dC-puromycin (F2P), a novel protein synthesis reporter based on the peptidyl transferase inhibitor puromycin [35] was used. Because puromycin is a structural analog of an amino-acyl tRNA molecule, it enters ribosomes actively engaged in translation where it becomes covalently attached to the carboxy-terminus of nascent proteins through a peptide linkage [36]. Initially, whether F2P can serve as a protein synthesis reporter in cultured hippocampal neurons was examined (FIGS. 11A, B). A brief (˜15 min) bath application of F2P resulted in fluorescence detected in both the cell body and the dendrites (FIG. 11A). The majority of the fluorescence observed in the dendrites reflects basal protein synthesis as it was significantly attenuated by co-application of anisomycin or unlabeled puromycin. When neurons were treated with the dopamine agonist SKF in the presence of F2P, a dramatic stimulation of protein synthesis in the cell body, dendrites and spines was observed [mean percent increase in F2P signal relative to control: 91.3+11.2%; n=14] (FIG. 11B). These data indicate that dopamine agonists can stimulate the synthesis of endogenous protein(s) in hippocampal neurons.

### Example 9

### Recording Excitatory Post Synaptic Currents in DHX Treated Hippocampal Neurons

Given the increase in the total and synaptic GluRl population, the effects of dopamine agonists on synaptic transmission was examined. To monitor synaptic strength before and after exposure to a dopamine agonist, miniature excitatory postsynaptic currents (mEPSCs) in cultured hippocampal neurons were examined. After a baseline recording period neurons were treated with DHX or DHX in the presence of anisomycin. It was observed that DHX induced a rapid increase in mEPSC frequency that was completely prevented when protein synthesis was inhibited. On average, DHX induced a 2-fold increase in mEPSC frequency. There was, however, no change in mEPSC amplitude elicited by the dopamine agonist. To determine whether the mEPSC frequency increase was due to a pre- or postsynaptic mechanism, the membrane impermeant PKA inhibitor peptide PKI₆₋₂₂ in the recording pipette was included. Blocking the activity of PKA postsynaptically completely prevented the DHX-induced increase in mEPSC frequency. These data indicate that activation of D1/D5 receptors induces a postsynaptically-driven increase in the frequency, but not amplitude, of mEPSCs.

Using both a GFP-based reporter of local translation and a novel, small molecule reporter, the stimulation of local protein synthesis in the dendrites of cultured hippocampal neurons by dopamine receptor agonists was observed. GluRl was identified as one synaptic protein whose synthesis is stimulated by dopamine receptor activation; dopamine agonists also induced an increase in surface GluRl, as has been observed in the nucleus accumbens [44, 45]. The agonist-stimulated increase in surface GluRl required new protein synthesis and increased the fraction of synapses that possess a surface GluRl cluster. The stimulated synthesis and surface expression of GluRl was accompanied by a dopamine agonist-stimulated increase in the frequency, but not amplitude, of mEPSCs. Because these changes occur rapidly (10-15 minutes), the data are most consistent with the idea that GluRl is locally synthesized. Indeed, two recent studies have demonstrated that glutamate receptors can be locally synthesized in dendrites [46, 47]. Taken together, these data suggest that D1/D5 receptor activation stimulates a local protein synthesis-dependent increase in surface GluRl at synaptic sites that did not previously possess functional postsynaptic GluRs, consistent with the activation of postsynaptically-silent synapses [48-51].

The data provide a potential cellular mechanism for the dopaminergic modulation of long-lasting plasticity at hippocampal synapses. Others have reported that dopamine or activators of the cAMP/PKA pathway can induce a long-lasting protein synthesis-dependent form of potentiation in hippocampal slices [31, 32]. It has also been shown that late-phase long-term potentiation (LTP) is diminished in hippocampal slices treated with dopamine receptor antagonists [52-54] or prepared from D1receptor knock-outs [55]. In addition, a PKA-dependent increase in GluRl synthesis has been observed during the late (3 hr post-induction) phase of LTP [56]. The data as disclosed indicate that dopamine may exert its effects on plasticity, at least in part, by local regulation of protein synthesis.

All of the compositions and methods disclosed and claimed herein can be made and executed without undue experimentation in light of the present disclosure. While the compositions and methods of this invention have been described in terms of illustrative embodiments, it will be apparent to those of skill in the art that variations may be applied to the composition, methods and in the steps or in the sequence of steps of the methods described herein without departing from the concept, spirit and scope of the invention. More specifically, it will be apparent that certain agents which are both chemically and physiologically related may be substituted for the agents described herein while the same or similar results would be achieved. Although the invention has been described with reference to the above examples, it will be understood that modifications and variations are encompassed within the spirit and scope of the invention.

### REFERENCES

1. The International Human Genome Sequencing Consortium, Nature, 2001, 409:860.
2. Venter, J. C., Adams, M. D., Myers, E. W., Li, P. W., Mural, R. J., Sutton, G. G., Smith, H. O., Yandell, M., Evans, C. A., Holt, R. A., et al., Science, 2001, 291:1304.
3. Duncan, R. F., Peterson, H., Hagedorn, C. H., and Sevanian, A., Biochem J, 2003, 369:213.
4. Lipschutz, J. H., Lingappa, V. R., and Mostov, K. E., J Biol Chem, 2003, 278:20954.
5. Constantinou, C., Bushell, M., Jeffrey, I. W., Tilleray, V., West, M., Frost, V., Hensold, J., and Clemens, M. J., Euro J Biochem, 2003, 270:3122.
6. Lockhart, D. L., and Winzeler, E. A., Nature, 2000, 405:827.
7. van Roessel, P., and Brand, A. H., Nat Cell Biol, 2002,4:E15.
8. Miyawaki, A., Llopis, J., Heim, R., McCaffery, J. M., Adams, J. A., Ikura, M., and Tsien, R. Y., Nature, 1997, 388:882.
9. Ting, A. Y., Kain, K. H., Klemke, R. L., and Tsien, R. Y., Proc Natl Acad Sci USA, 2001, 98:15003.
10. Steward, O., and Schuman, E. M., Annu Rev Neurosci, 2001, 24:299.
11. Aakalu, G., Smith, W. B., Nguyen, N., Jiang, C. G., and Schuman, E. M., Neuron, 2001, 30:489.
12. Yarmolinsky, M. B., and de la Haba, G., Proc Natl Acad Sci USA, 1959, 45:721.
13. Nathans, D., Proc Natl Acad Sci USA, 1964, 51:585.
14. Starck, S. R., and Roberts, R. W., RNA, 2002, 8:890.
15. Nemota, N., Miyamoto-Sato, M., and Yanagawa, H., FEBS Lett, 1999, 462:43.
16. Doi, N., Takashima, H., Kinjo, M., Sakata, K., Kawahashi, Y., Oishi, Y., Oyama, R., Miyamoto-Sato, E., Sawasaki, T., Endo, Y., and Yanagawa, H., Genome Res, 2002, 12:487.
17. Kawahashi, Y., Doi, N., Takashima, H., Tsuda, C., Oishi, Y., Oyama, R., Yonezawa, M., Miyamoto-Sato, E., and Yanagawa, H., Proteomics, 2003, 3:1236.
18. Starck, S. R., Qi, X., Olsen, B. N., and Roberts, R. W., J Am Chem Soc, 2003, 125:8090.
19. Diamond, R. A., and DeMaggio, S., eds. In Living Color: Protocols in Flow Cytometry and Cell Sorting, 2000, Springer-Verlag: Berlin.
20. Van Parijs, L., Refaeli, Y., Lord, J. D., Nelson, B. H., Abbas, A. K., and Baltimore, D., Immunity, 1999, 11:281.
21. de la Luna, S., and Ortin, J., Methods Enzymol, 1992, 216:376.
22. Porter, J. N., Hewitt, R. I., Hesseltine, C. W, Krupka, G., Lowery, J. A., Wallace, W. S., Bohonos, N., and Williams, J. H., Antibiotics and Chemo, 1952, 11:409.
23. Pérez-González, J. A., Var,a J., and Jiménez, A., Biochem Biophys Res Commun, 1983, 113:772.
24. Bain, G., Quong, M. W., Soloff, R. S., Hedrick, S. M., and Murre, C., J Exp Med, 1999, 190:1605.
25. Wolin, S. L, and Walter, P., EMBO J, 1988, 7:3559.
26. Iborra, F. J., Jackson, D. A., and Cook, P. R., Science, 2001, 293:1139.
27. Nathanson, L., Xia, T., and Deutscher, M. P., RNA, 2003, 9:9.
28. Lelouard, H., Ferrand, V., Marguet, D., Bania, J., Camosseto, V., David, A., Gatti, E., and Pierre, P., J Cell Biol, 2004, 164:667.
29. Schagger, H., and von Jagow, G. V., Anal Biochem, 1987, 166:368.
30. Naviaux, R. K., Costanzi, E., Haas, M., and Verma, I. M., J Virology, 1996,70:5701.
31. Frey, U., Huang, Y. Y., and Kandel, E. R., Science, 1993, 260:1661.
32. Huang, Y. Y., Kandel, E. R., Proc Natl Acad Sci USA, 1995, 92, 2446.
33. Bach, M. E., et al., Proc Natl Acad Sci USA, 1999, 96, 5280.
34. Aakalu, G., Smith, W. B., Jiang, C., Nguyen, N., and Schuman, E. M., Neuron, 2001, 30:489.
35. Starck, S. R., and Roberts, R. W., RNA, 2002, 8:890.
36. Nathans, D., Proc Natl Acad Sci USA, 1964, 51:585.
37. Steward, O., and Levy, W. B., J Neurosci, 1982, 2:284.
38. Torre, O. and Steward, E. R., J Neurosci, 1992, 12:762.
39. Kang, H. and Schuman, E. M., Science, 1996, 273:1402.
40. Martin, K. C., et al., Cell, 1997, 91:927.
41. Huber, K. M., Kayser, M. S., and Bear, M. F., Science, 2000, 288:1254.
42. Bouron, A. and Reuter, H., Neuroscience, 1999, 94:1063.
43. Otmakhova, N. A. and Lisman, J. E., J Neurosci, 1996, 16:7478.
44. Chao, S. Z., Ariano, M. A., Peterson, D. A., and Wolf, M. E., J Neurochem, 2002, 83:704.
45. Mangiavacchi, W. M., J Neurochem, 2004, 88:1261.
46. Kacharmina, J. E., Job, C., Crino, P., and Eberwine, J., Proc Natl Acad Sci USA, 2000, 97:11545.
47. Ju, W., et al., Nat Neurosci, 2004, 7:244.
48. Isaac, J. T., Nicoll, R. A., and Malenka, R. C., Neuron, 1995, 15:427.
49. Liao, S., Hessler, N. A., and Malinow, R., Nature, 1995, 375, 400.
50. Liao, D., Zhang, X., O'Brien, R., Ehlers, M. D., and Huganir, R. L., Nat Neurosci, 1999, 2:37.
51. Petralia, R. S., et al., Nat Neurosci, 1999, 2:31.
52. Frey, U., Matthies, H., and Reymann, K. G., Neurosci Lett, 1991, 129:111.
53. Frey, U., Schroeder, H., and Matthies, H., Brain Res, 1990, 522:69.
54. Swanson-Park, J. L., et al., Neuroscience, 1999, 92:485.
55. Matthies, H., et al., Neuroreport, 1997, 8:3533.
56. Nayak, A., Zastrow, D. J., Lickteig, R., Zanister, N. R., and Browning, M. D., Nature, 1998, 394:690.
57. Richmond, S. A., et al., Neuroscience, 1996, 75:69.
58. Patrick, G. N., Bingol, B., Weld, H. A., and Schuman, E. M., Curr Biol, 2003, 13:2073.

Accordingly, the invention is limited only by the following claims.

What is claimed is:

1. A labeled protein comprising a C-terminal chemically linked to a conjugate, wherein the conjugate comprises a phosphonate-puromycin-aminonucleoside-R₃ compound or phosphonate-puromycin-aminonucleoside-R₃ compound derivative linked to at least one label moiety, which R₃ is an amino acid or an amino acid analog.

2. The protein of claim 1, wherein the compound or compound derivative is of the general Formula I: 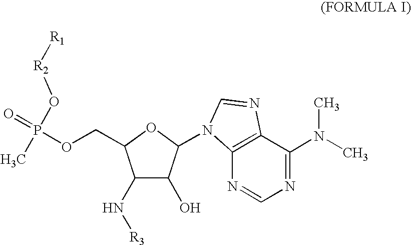
wherein R₁ is one or more label moieties;
R₂ is a nucleotide; and
and R₃ is:

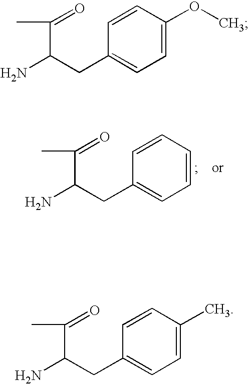

3. The protein of claim 2, wherein R₃ is:

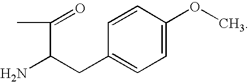

4. The protein of claim 2, wherein R₂ is a ribonucleotide or deoxyribonucleotide.

5. The protein of claim 2, wherein R₂ is deoxycytidine-5′-monophosphate.

6. The protein of claim 2, wherein R₁ is a fluorescent substance, biotin, protein, peptide, nucleic acid, sugar, lipid, or dye.

7. The protein of claim 1, wherein the label moiety comprises a dimethoxytrityl (DMT) moiety.

8. The protein of claim 1, wherein the conjugate comprises two or more label moieties.

9. The protein of claim 8, wherein at least one moiety is a fluorescent substance.

10. The protein of claim 9, wherein the fluorescent substance is selected from the group consisting of the fluorescein series.

11. The protein of claim 9, wherein the fluorescent substance is selected from the group consisting of the Cy series.

12. The protein of claim 1 or 9, wherein the conjugate comprises biotin.

13. A method of monitoring protein expression comprising:
a) contacting a sample comprising the minimum components necessary for protein translation with a conjugate comprising a phosphonate-puromycin-aminonucleoside-R₃ compound or phosphonate-puromycin-aminonucleoside-R₃ compound derivative linked to at least one label moiety under conditions that allow for protein translation, wherein R₃ is an amino acid or an amino acid analog; and
b) determining the presence of label incorporated into protein after a sufficient time, wherein incorporation of label into protein is correlated with protein expression.

14. The method of claim 13, wherein R₃ is:

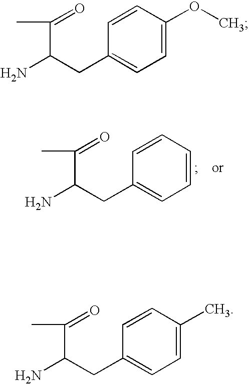

15. The method of claim 14, wherein R₃ is:

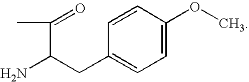

16. The method of claim 13, wherein the sample is an in vitro translation extract or a cell.

17. The method of claim 16, wherein the cell is transfected with a fluorescence based reporter vector.

18. The method of claim 13, wherein the determining step further comprises chromatograpy, blotting, spectrometry, microscopy, flow cytometry, imaging, immunochemistry, or combinations thereof.

19. The method of claim 13, further comprising resolution of temporal protein expression and/or spatial protein expression.

20. The method of claim 13, wherein the structure of the conjugate is a X—N-phosphonate-puromycin-aminonucleoside-R₃ compound or X—N-phosphonate-puromycin-aminonucleoside-R₃ compound derivative, which X is a label moiety and N is a ribonucleotide or deoxyribonucleotide.

21. The method of claim 20, wherein N is deoxycytidine-5′-monophosphate.

22. The method of claim 20, wherein X is a fluorescent substance, biotin, protein, peptide, nucleic acid, sugar, lipid, or dye.

23. The method of claim 20, wherein X comprises a dimethoxytrityl (DMT) moiety.

24. The method of claim 13, wherein the conjugate comprises two or more label moieties.

25. The method of claim 13, wherein the conjugate has an IC₅₀ range from between about less than 1 μM to about 30 μM.

26. A method of identifying a protein modulated by an exogenous agent comprising:
a) contacting an exogenous agent with a sample comprising the minimum components necessary for protein translation;
b) contacting the sample of step (a) with a conjugate comprising a phosphonate-puromycin-aminonucleoside-R₃ compound or phosphonate-puromycin-aminonucleoside-R₃ compound derivative linked to at least one label under conditions that allow for protein translation, wherein R₃ is an amino acid or an amino acid analog;
c) determining the presence of label incorporated into a protein after a sufficient time; and
d) comparing protein incorporation patterns in the presence and absence of the exogenous agent, wherein changes in incorporation of label for a protein in the sample in the presence and absence of the exogenous agent correlate with modulation of such protein by the agent.

27. The method of claim 26, wherein R₃ is:

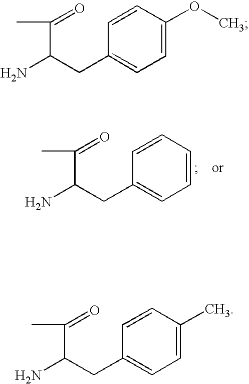

28. The method of claim 27, wherein R₃ is:

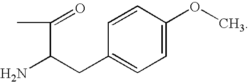

29. The method of claim 26, wherein the sample is an in vitro translation extract or a cell.

30. The method of claim 29, wherein the cell is transfected with a fluorescence based reporter vector.

31. The method of claim 26, wherein the exogenous agent is a mineral, ion, gas, light, sound, small molecule, agonist, antagonist, amino acid, ligand, receptor, protein, peptide, antibody, nucleic acid, lipid, carbohydrate, cell, tissue, virus, organ, bodily fluid, buffer, media, conditioned media, temperature, pressure or a combination thereof.

32. The method of claim 26, wherein the structure of the conjugate is X—N-phosphonate-puromycin-aminonucleoside-R₃ compound or X—N-phosphonate-puromycin-aminonucleoside-R₃ compound derivative, which X is a label moiety and N is a ribonucleotide or deoxyribonucleotide.

33. The method of claim 32, wherein N is deoxycytidine-5′-monophosphate.

34. The method of claim 32, wherein X is a fluorescent substance, biotin, protein, peptide, nucleic acid, sugar, lipid or dye.

35. The method of claim 32, wherein X comprises a dimethoxytrityl (DMT) moiety.

36. The method of claim 26, wherein the conjugate comprises two or more label moieties.

37. The method of claim 26, wherein the conjugate has an IC₅o range from between about less than 1 μM to about 30 μM.

38. A conjugate comprising a phosphonate-puromycin-aminonucleoside-R₃ compound or phosphonate-puromycin-aminonucleoside-R₃ compound derivative linked to at least two label moieties, wherein R₃ is an amino acid or amino acid analog.

39. The conjugate of claim 38, wherein R₃ is:

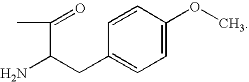

40. The conjugate of claim 38, wherein the phosphonate moiety is chemically linked to a ribonucloetide or deoxyribonucleotide.

41. The conjugate of claim 38, which comprises the formula 2X—N-puromycin compound or 2X—N-puromycin compound derivative, wherein 2× represents the label moieties and N is a ribonucleotide or a deoxyribonucleotide.

42. The conjugate of claim 38, wherein the conjugate has an IC₅₀ range from between about less than 1 μM to about 30 μM.

43. A kit comprising:
a) a conjugate comprising a phosphonate-puromycin-aminonucleoside-R₃ or phosphonate-puromycin-aminonucleoside-R₃ derivative linked to at least one label moiety, wherein R₃ is an amino acid or an amino acid analog;
b) instructions containing method steps for practicing identifying a protein modulated by an exogenous agent, monitoring protein expression, labeling a protein at a C-terminal, or a combination thereof; and
c) container comprising reagents necessary for carrying out the methods of component (b).

44. The kit of claim 43, wherein R₃ is:

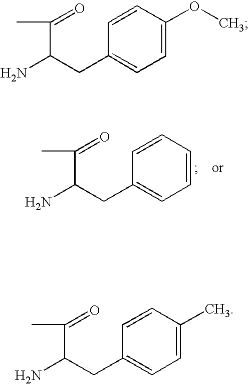

45. The kit of claim 44, wherein R₃ is;

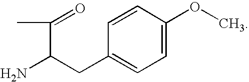

46. The kit of claim 43, wherein the kit conjugate comprises two or more moieties.

47. The kit of claim 43, further comprising a fluorescence based vector.
